(19) United States
(12) Patent Application Publication
(10) Pub. No.: US 2003/0181803 A1
(43) Pub. Date: Sep. 25, 2003

(54) Surgical microscope with information system

(75) Inventor: Ulrich Sander, Rebstein (CH)

Correspondence Address: DAVIDSON, DAVIDSON & KAPPEL, LLC, 485 SEVENTH AVENUE, 14TH FLOOR, NEW YORK, NY, 10018, US

(73) Assignee: Leica Microsystems (Schweiz) AG, Heerbrugg (CH)

(21) Appl. No.: 10/393,779

(22) Filed: Mar. 21, 2003

(30) Foreign Application Priority Data
Mar. 22, 2002 (DE) DE 102 12 805.7

(51) Int. Cl. A61B 5/05

(52) U.S. Cl. 600/407

(57) ABSTRACT

A surgical microscope, for example an ophthalmic surgical microscope, includes an information system having a data superimposition module and an electronics unit for ascertaining and displaying patient data. A program-controlled connection between the electronics unit and the data superimposition module transmits the displayed parameters from the electronics unit to the data superimposition module, which superimposes those parameters into a user's field of view.

This application claims priority to German patent application 102 12 805.7, which is hereby incorporated by reference herein.

### BACKGROUND

The invention relates to a surgical microscope having an information system that includes a data superimposition module and an electronics unit for ascertaining and displaying patient data, for example irradiation data for the patient, that are measured in the microscope system.

It is known that in surgical microscopes used in ophthalmology, the light for illumination of the patient's eye can cause retinal damage. It is therefore advisable to measure, monitor, and record certain parameters of the illumination. These parameters are, for example, the light intensity of the illumination, the irradiation time, and the irradiation dose.

The light intensity of the illumination is normally depicted visibly, generally by way of a display module on the control electronics and/or by means of a marking on a potentiometer for adjusting the light intensity. The irradiation time is measured by means of a time recording system (clock), and the irradiation dose is ascertained by the surgeon from the two parameters of light intensity and irradiation time.

A dose measurement system in combination with a surgical microscope is described in U.S. Pat. No. 4,657,013 and U.S. Pat. No. 4,682,595.

The irradiation time and irradiation dose could also be displayed on a separate unit, or also forwarded to the control electronics of the microscope and displayed in simple fashion there.

Company publications of Leica Microsystems AG have already disclosed the technical implementation of superimpositions into the surgeon's field of view for various purposes—in particular for video, endoscopic, or patient-data overlays—using the so-called “imaging module” (“The Leica Imaging Module,” publication date October 1999) or its successor model the DI module (“The Leica Dual Imaging and the Ultra Observer,” published 2001). Further information is contained in WO 01/27659 A2 and WO 01/48528 A2, and in previously undisclosed German patent application having the official application number 101 57 613.7.

These modules are placed on the surgical microscope between the zoom system and the observer tube. The parameters to be displayed are sent, for example from a measurement or endoscopic unit, to the imaging module, specifically via an electrical connecting cable or via light guide (cf. EP 102 901.6) or by broadcast (cf. previously undisclosed German patent application having official application number 102 02 125.2). The video or patient data presented in the display of these modules are thus displayed in the surgeon's field of view.

As another variant, the data are superimposed not into the intermediate image plane between the zoom system and observer tube, but directly into the eyepiece.

### SUMMARY OF THE INVENTION

The above known systems exhibit, among others, the following disadvantages:

Since the control electronics of the microscope are located far from the surgeon's field of view, and since the surgeon cannot continuously keep the displays in view, the location of the displays is impractical for actual use; additional persons must monitor the displays and report verbally to the surgeon.

Because the surgeon looks in an alternating fashion between the display modules and the surgical field, accommodation problems are caused for the surgeon's eye and are in any event time-consuming.

At present, the aforementioned data are not all displayed directly on the control electronics of the microscope, but rather are in some cases shown in separate devices.

Although the surgeon can be made aware of safety-relevant data by way of acoustic signals, he or she must then nevertheless look away from the surgical field in order to note and interpret the current status, at least for monitoring purposes.

Continuous monitoring as to how much of the permissible radiation dose has already been administered is also possible only by turning the surgeon's view away from the surgical field toward the display of the dose measurement device, or by involving additional (auxiliary) personnel.

It is consequently an object of the present invention to provide an information system for a surgical microscope that eliminates the aforesaid drawbacks and therefore permits the surgeon to work more safely and more quickly.

The present invention provides a surgical microscope, for example a (stereoscopic) ophthalmic surgical microscope, having an information system, which encompasses a data superimposition module (5) and an electronics unit (6) for ascertaining and displaying patient data. A program-controlled connection (12) is provided between the electronics unit (6) and the data superimposition module (5) and, in the operating state, transmits the displayed parameters from the electronics unit (6) to the data superimposition module (5). The data superimposition module (5) superimposes those parameters into a user's field of view.

The parameters displayed on an electronics unit, for example for light intensity, irradiation time and/or irradiation dose, are processed by means of an electronics unit and transferred, according to the present invention, to a known data superimposition module. The latter superimposes the parameters into the surgeon's field of view. Transfer from the electronics unit to the data superimposition module can be accomplished in conventional electrical fashion, or also by means of glass fibers or broadcast systems, in which context “broadcast systems” encompass electromagnetic waves, e.g., infrared or radio waves, as well as acoustic waves.

In addition, according to the present invention, threshold or limit values can also be superimposed into the surgeon's field of view.

The data superimposition module thus, according to the present invention, continuously or selectably superimposes into the surgeon's field of view all the parameters relevant for irradiation of the eye.

According to an embodiment, display of the data can also be controlled in such a way that when safety-relevant data are blanked out, the system automatically delivers those data into the surgeon's field of view if a threshold happens to be reached or even exceeded. According to the present invention, for example, a “Clock” symbol blinks if the irradiation time is exceeded.

Advantages of the new information system in a surgical microscope described above include the following:

Limitations are not imposed on surgical activity as a result of the surgeon diverting his or her view from the surgical field.

There are no accommodation problems for the surgeon's eye.

Safety-relevant parameters can, if necessary, be monitored continuously and by the surgeon himself.

The surgeon can more effectively schedule his surgical technique and therefore better protect the patient's eye, since the surgeon can view the irradiation parameters at any time during the operation.

### BRIEF DESCRIPTION OF THE DRAWING

FIG. 1 schematically depicts a surgical microscope having an information system according to the present invention.

### DETAILED DESCRIPTION

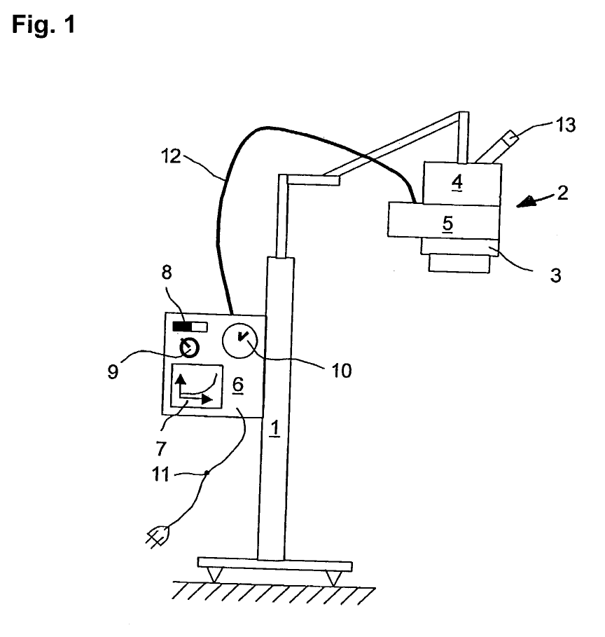

FIG. 1 shows a microscope 2 mounted on a stand 1, having a zoom 3, a tube 4, an eyepiece 13, a data superimposition module 5, and an electronics unit 6. Electronics unit 6 encompasses, for example, a dose display 7, a light intensity display 8, a potentiometer 9 for adjusting light intensity, a time recording unit 10, and a power connection 11.

The parameters presented on electronics unit 6 are processed therein and conveyed by means of a data transfer connection 12 to data superimposition module 5, and superimposed into the surgeon's field of view, as necessary, using data superimposition module 5.

According to an embodiment, the desired values, actions, and/or parameters—for example superimposition, blanking, shutoff, auto-mode—are selected by the surgeon using an eye-control system, for example with the apparatus described in EP 788 613 B1, or using a voice-control system.

What is claimed is:

1. An surgical microscope comprising:
a data superimposition module;
an electronics unit configured to ascertain and display patient data; and
a program-controlled connection connecting the electronics unit and the data superimposition module, the program-controlled connection being configured to transmit, in an operating state, the patient data from the electronics unit to the data superimposition module;
wherein the data superimposition module is configured to superimpose the patient data into a field of view of a user.

2. The surgical microscope as recited in claim 1 wherein the data superimposition module, the electronics unit and the program-controlled connection form at least a portion of an information system.

3. The surgical microscope as recited in claim 1 wherein the surgical microscope is a stereoscopic ophthalmic surgical microscope.

4. The surgical microscope as recited in claim 1 wherein the patient data include at least one of an irradiation light intensity, an irradiation time and an irradiation dose.

5. The surgical microscope as recited in claim 1 wherein the electronics unit is configured to enable a superimposing of at least one of comparative values and threshold values into the field of view using the data superimposition module.

6. The surgical microscope as recited in claim 1 wherein the program-controlled connection includes at least one of a transfer cable, a light guide, and a broadcast system.

7. The surgical microscope as recited in claim 1 wherein the patient data include at least one of an irradiation light intensity, an irradiation time and an irradiation dose, and wherein the data superimposition module is configured to selectably superimpose the patient data into the field of view continuously and discontinuously.

8. The surgical microscope as recited in claim 1 wherein the patient data include at least one of an irradiation light intensity, an irradiation time and an irradiation dose, and wherein the electronics unit is configured to set threshold values for at least one of the illumination intensity, the irradiation time and the irradiation dose.

9. The surgical microscope as recited in claim 1 wherein the electronics unit is configured to cause a display to automatically switch on and deliver at least one of a visual and an acoustic warning signal upon an exceeding of a threshold value.

10. The surgical microscope as recited in claim 1 further comprising an eye-control system configured to select a desired mode using an eye movement of the user.

11. The surgical microscope as recited in claim 10 wherein the desired mode includes at least one of a superimposition, a blanking, a shutoff, and an automatic mode.

12. The surgical microscope as recited in claim 1 further comprising a voice-control system configured to select a desired mode using a recognition of a voice of the user.

13. The surgical microscope as recited in claim 12 wherein the desired mode includes at least one of a superimposition, a blanking, a shutoff, and an automatic mode.

14. An information system for a surgical microscope, the information system comprising:
a data superimposition module;
an electronics unit configured to ascertain and display patient data; and
a program-controlled connection connecting the electronics unit and the data superimposition module, the program-controlled connection being configured to transmit, in an operating state, the patient data from the electronics unit to the data superimposition module;
wherein the data superimposition module is configured to superimpose the patient data into a field of view of a user.

15. The information system as recited in claim 14 wherein the surgical microscope is a stereoscopic ophthalmic surgical microscope.

16. The information system as recited in claim 14 wherein the patient data include at least one of an irradiation light intensity, an irradiation time and an irradiation dose.

17. The information system as recited in claim 14 wherein the electronics unit is configured to enable a superimposing of at least one of comparative values and threshold values into the field of view using the data superimposition module.

18. The information system as recited in claim 14 wherein the program-controlled connection includes at least one of a transfer cable, a light guide, and a broadcast system.

19. The information system as recited in claim 14 wherein the patient data include at least one of an irradiation light intensity, an irradiation time and an irradiation dose, and wherein the data superimposition module is configured to selectably superimpose the patient data into the field of view continuously and discontinuously.

20. The information system as recited in claim 14 wherein the patient data include at least one of an irradiation light intensity, an irradiation time and an irradiation dose, and wherein the electronics unit is configured to set threshold values for at least one of the illumination intensity, the irradiation time and the irradiation dose.
